(19) United States
(12) Patent Application Publication
(10) Pub. No.: US 2010/0234592 A1
(43) Pub. Date: Sep. 16, 2010

(54) Corrosion Inhibitors Having Increased Biodegradability and Reduced Toxicity

(76) Inventors: Dirk Leinweber, Kelkheim (DE); Michael Feustel, Kongernheim (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 12/223,055

(22) PCT Filed: Jan. 10, 2007
(86) PCT No.: PCT/EP2007/000140

§ 371 Date: Sep. 9, 2008

(30) Foreign Application Priority Data
Jan. 20, 2006 (DE) 10 2006 002 784.1

(51) Int. Cl. C07D 295/037; C07C 233/47

(52) U.S. Cl. 544/110; 554/42

(57) ABSTRACT

The invention relates to the use of salts of compounds of the formula (1)

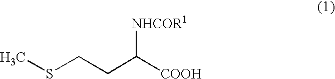

and amines of the formula (2)

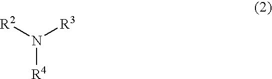

where R¹ is C₁- to C₃₀-alkyl, C₂- to C₃₀-alkenyl, C₆- to C₃₀-aryl or C₇- to C₃₀-alkylaryl, R² is C₁- to C₃₀-alkyl, C₂- to C₃₀-alkenyl, C₆- to C₃₀-aryl or C₇- to C₃₀-alkylaryl, or an optionally heteroatom containing organic radical having from 1 to 30 carbon atoms, and R³ and R⁴ are each independently hydrogen, C₁- to C₃₀-alkyl, C₂- to C₃₀-alkenyl, C₆- to C₃₀-aryl or C₇- to C₃₀-alkylaryl, or an optionally heteroatom containing organic radical having from 1 to 30 carbon atoms, where R³ and R⁴ can also form, with the inclusion of the nitrogen atom, a ring having from 5 to 7 ring atoms.

The present invention relates to a process for inhibiting corrosion on and in devices for extraction and transport of hydrocarbons in mineral oil extraction and processing by adding a salt of a nitrogen base and an N-acylmethionine to the corrosive system.

In industrial processes in which metals come into contact with water or else with oil-water biphasic systems, there is the risk of corrosion. This is particularly marked when the aqueous phase, as in the case of mineral oil extraction and processing processes, has a high salt content or is acidic as a result of dissolved acidic gases such as carbon dioxide or hydrogen sulfide. The exploitation of a deposit and the processing of mineral oil are therefore impossible without specific additives to protect the equipment used.

Although suitable anticorrosives for mineral oil extraction and processing have been known for some time, they will be unacceptable in the future for offshore applications for reasons of environmental protection.

As typical prior art corrosion inhibitors, amides, amidoamines or imidazolines of fatty acids and polyamines have exceptionally good oil solubility and are therefore present only in a low concentration in the corrosive water phase owing to poor partitioning equilibria. Accordingly, these products have to be used in high dosage in spite of their poor biodegradability.

Quaternary alkylammonium compounds (quats) are alternative prior art anticorrosives which, as well as the corrosion-inhibiting properties, may also possess biostatic properties. In spite of an improved water solubility, the quats, for example compared to the imidazolines, exhibit a significantly reduced film persistence and therefore likewise lead to effective corrosion protection only in a relatively high dosage. The high algal toxicity and the moderate biodegradability are restricting the use of quats ever more to ecologically insensitive fields of use.

U.S. Pat. No. 4,240,823 describes N-acylmethionine derivatives which are used as growth regulators in the field of crop protection. Amine salts of N-acylmethionine derivatives are not described.

JP-A-8 337 562 and JP-A-8 337 563 describe N-acylamino acids and their alkali metal salts, which can also be used as corrosion inhibitors. No amine salts of N-acylmethionine derivatives are described.

JP-A-49 026 145 describes N-acylamino acid alkali metal salts which can be used as corrosion inhibitors. As an example, N-lauroylglycine sodium salt is mentioned. Amine salts of N-acylmethionine derivatives are not described.

However, a disadvantage of the prior art compounds is that their effectiveness is insufficient and that they have a high tendency to foam.

It was an object of the present invention to find novel corrosion inhibitors which, with constantly good or improved corrosion protection, as well as a good water solubility and low foam formation, also offer improved biodegradability and lower toxicity compared to the prior art corrosion inhibitors.

It has now been found that, surprisingly, N-acylmethionine-ammonium salts have outstanding action as corrosion inhibitors and a low foam formation tendency, and also good biodegradability and reduced toxicity.

The invention thus provides for the use of salts of compounds of the formula (1)

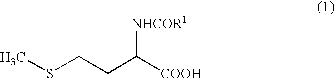

and amines of the formula (2)

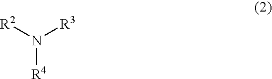

in which
R¹ is C₁- to C₃₀-alkyl, C₂- to C₃₀-alkenyl, C₆- to C₃₀-aryl or C₇- to C₃₀-alkylaryl,
R² is C₁- to C₃₀-alkyl, C₂- to C₃₀-alkenyl, C₆- to C₃₀-aryl or C₇- to C₃₀-alkylaryl, or an organic radical which optionally contains heteroatoms and has from 1 to 30 carbon atoms, and
R³, R⁴ are each independently hydrogen, C₁- to C₃₀-alkyl, C₂- to C₃₀-alkenyl, C₆- to C₃₀-aryl or C₇- to C₃₀-alkylaryl, or an organic radical which optionally contains heteroatoms and has from 1 to 30 carbon atoms, where R³ and R⁴ may also form a cycle with from 5 to 7 ring atoms including the nitrogen atom, as corrosion inhibitors.

The invention further provides a process for inhibiting corrosion on metal surfaces, especially of iron-containing metals, by adding at least one salt of compounds of the formulae (1) and (2) to a corrosive system which is in contact with the metal surfaces.

The invention further provides salts obtainable by the reaction of at least one compound of the formula (1) with at least one compound of the formula (2)

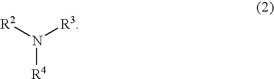

Corrosive systems in the context of this invention are preferably liquid/liquid or liquid/gaseous polyphasic systems consisting of water and hydrocarbons which comprise corrosive constituents, such as salts and acids, in free and/or dissolved form. The corrosive constituents may also be gaseous, for instance hydrogen sulfide and carbon dioxide.

Hydrocarbons in the context of this invention are organic compounds which are constituents of mineral oil/natural gas, and conversion products thereof. Hydrocarbons in the context of this invention are also volatile hydrocarbons, for example methane, ethane, propane, butane. For the purposes of this invention, they also include the further gaseous constituents of mineral oil/natural gas, for instance hydrogen sulfide and carbon dioxide.

The invention further provides for the use of the compounds of the formulae (1) and (2) as metal processing agents. In this context, the inventive compounds offer very good corrosion protection even in the case of high mechanical stress, such as in the course of sanding, cutting and drilling of metal workpieces.

In a preferred embodiment of the invention, the compound of the formula (2) is a cyclic amine of the formula (3)

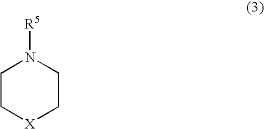

in which
R⁵ is hydrogen or a C₁₋₃₀ alkyl radical which optionally contains heteroatoms, and
X is C, O or N.

R¹ is preferably an alkyl or alkenyl group having from 2 to 24 carbon atoms, especially an alkyl or alkenyl group having from 8 to 18 carbon atoms.

R² is an organic radical which may contain from 1 to 30 carbon atoms and optionally heteroatoms. When R² contains heteroatoms, they are preferably nitrogen atoms and/or oxygen atoms. In a preferred embodiment, R² is —CH₂—CH₂—OH.

R³ and R⁴ may each independently be any organic radicals which contain hydrogen or from 1 to 30 carbon atoms and optionally heteroatoms. When R³ and/or R⁴ contain heteroatoms, they are preferably nitrogen and/or oxygen atoms. In a preferred embodiment, one or both R³ and R⁴ radicals are —CH₂—CH₂—OH. The formula (2) thus preferably represents mono-, di- or triethanolamine. Also in accordance with the invention is the use of alkoxylated alkanolamines, for example of ethoxylated N,N-dibutylamino-ethanol.

In a further preferred embodiment of the invention, R¹ is C₂- to C₃₀-alkyl, C₂- to C₃₀-alkenyl, C₆- to C₃₀-aryl or C₇- to C₃₀-alkylaryl, and R² is C₁- to C₃₀-alkyl, C₂- to C₃₀-alkenyl, C₆- to C₃₀-aryl or C₇- to C₃₀-alkylaryl, or an organic radical which optionally contains nitrogen atoms and has from 1 to 30 carbon atoms.

The inventive compounds may be used alone or in combination with other known corrosion inhibitors. In general, an amount of the inventive corrosion inhibitor sufficient to obtain sufficient corrosion protection under the given conditions will be used.

Preferred use concentrations of the corrosion inhibitors based on the pure inventive salts are from 5 to 5000 ppm, preferably from 10 to 1000 ppm, especially from 15 to 150 ppm.

Particularly suitable corrosion inhibitors are also mixtures of the inventive salts with other corrosion inhibitors and/or prior art corrosion inhibitors.

Particularly suitable corrosion inhibitors and thus a preferred embodiment of this invention are mixtures of the inventive salts with amidoamines and/or imidazolines formed from fatty acids and polyamines and salts thereof, quaternary ammonium salts, oxyethylated and/or oxypropylated amines, amphoglycinates and -propionates, betaines or compounds described in DE-A-199 30 683.

N-Acylmethionine derivatives are prepared by acylating methionine by means of a carbonyl chloride or carboxylic anhydride in the presence of a base (e.g. sodium hydroxide). By subsequent neutralization, removal of the aqueous salt solution and reaction with amines, the inventive N-acylmethionine ammonium salts are preparable.

For this purpose, preference is given for economic reasons to using DL-methionine which can, though, likewise be used in enantiomerically pure forms.

For the acylation, preference is given to using C₈₋₁₈ alkyl or alkenyl chlorides, for example octanoyl chloride, decanoyl chloride, dodecanoyl chloride, coconut fatty acid chloride or oleyl chloride.

Amines of the formula (2) used with preference are, for example, methylamine, ethylamine, propylamine, butylamine, cyclohexylamine, dicyclohexylamine, laurylamine, coconut fatty amine, stearylamine, dimethylamine, diethylamine, dipropylamine, dibutylamine, 3-dimethylaminopropylamine, 3-diethylaminopropylamine, 3-morpholinopropylamine, diethylenetriamine, triethylenetetramine, tetraethylenepentamine, monoethanolamine, diethanolamine, triethanolamine, morpholine, morpholine production residues, N,N-dimethylaminoethanol, N,N-diethylaminoethanol, N,N-dibutylamino-ethanol, 3-dimethylaminopropanol, N-hydroxyethylmorpholine, 3-amino-propanol, isopropanolamine, 2-(2-aminoethoxy)ethanol and cyclohexyl-amino-N,N-diethanol, aminoethylmorpholine and aminoethylpiperazine.

### EXAMPLES

### General Method for the Preparation of N-Acylmethionine Ammonium Salts

In a standard stirred apparatus, 1 mol of DL-methionine in 300 ml of water is neutralized with 50% sodium hydroxide solution. 1 mol of carbonyl chloride is metered at 15-20° C. into the solution formed, in the course of which the pH is kept at 10-13 by parallel metered addition of 15% sodium hydroxide solution. The reaction solution is stirred at room temperature for 3 h. The N-acyl-DL-methionine sodium salt formed is then neutralized with 32% hydrochloric acid, removed from the aqueous salt phase and dried. Subsequently, the N-acyl-DL-methionine is converted to the N-acyl-DL-methionine ammonium salt by adding an equimolar amount of the appropriate amine. The resulting product is characterized by means of acid number (AN) and basic nitrogen (bas. N). Percentages are percentages by weight based on the weight of the inventive salt.

### Example 1

### N-Octyl-DL-methionine monoethanolammonium salt

162.7 g of octanoyl chloride, 117.2 g of DL-methionine and 61.1 g of monoethanolamine were used to obtain 304.5.1 g of N-octyl-DL-methionine monoethanolammonium salt with AN=184 mg KOH/g and bas. N=4.58%.

### Example 2

### N-Octyl-DL-methionine triethanolammonium salt

162.7 g of octanoyl chloride, 117.2 g of DL-methionine and 149.2 g of triethanolamine were used to obtain 392.0 g of N-octyl-DL-methionine triethanolammonium salt with AN=143 mg KOH/g and bas. N=3.55%.

### Example 3

### N-Dodecyl-DL-methionine cyclohexylammonium salt

218.8 g of dodecanoyl chloride, 1172 g of DL-methionine and 99.2 g of cyclohexylamine were used to obtain 398.4 g of N-dodecyl-DL-methionine cyclohexylammonium salt with AN=140 mg KOH/g and bas. N=3.49%.

### Example 4

### N-Dodecyl-DL-methionine dibutylammonium salt

218.8 g of dodecanoyl chloride, 117.2 g of DL-methionine and 129.3 g of dibutylamine were used to obtain 428.3 g of N-dodecyl-DL-methionine dibutylammonium salt with AN=130 mg KOH/g and bas. N=3.23%.

### Example 5

### N-Cocoyl-DL-methionine morpholinium salt

225.3 g of coconut fatty acid chloride, 117.2 g of DL-methionine and 87.1 g of morpholine were used to obtain 392.0 g of N-cocoyl-DL-methionine morpholinium salt with AN=142 mg KOH/g and bas. N=3.55%.

### Example 6

### N-Cocoyl-DL-methionine N,N-diethyl-(2-hydroxyethyl)-ammonium salt

225.3 g of coconut fatty acid chloride, 117.2 g of DL-methionine and 117.2 g of N,N-diethylaminoethanol were used to obtain 419.5 g of N-cocoyl-DL-methionine N,N-diethyl-(2-hydroxyethyl)ammonium salt with AN=134 mg KOH/g and bas. N=3.30%.

### Example 7

### N-Oleyl-DL-methionine 2-(2-hydroxyethoxy)ethylammonium salt

300.9 g of oleyl chloride, 117.2 g of DL-methionine and 105.4 g of 2-(2-aminoethoxy)ethanol were used to obtain 482.7 g of N-oleyl-DL-methionine 2-(2-hydroxyethoxy)ethylammonium salt with AN=116 mg KOH/g and bas. N=2.87%.

### Example 8

### N-Oleyl-DL-methionine triethanolammonium salt

300.9 g of oleyl chloride, 117.2 g of DL-methionine and 149.2 g of triethanolamine were used to obtain 526.0 g of N-oleyl-DL-methionine triethanolammonium salt with AN=106 mg KOH/g and bas. N=2.64%.

### Effectiveness of the Inventive Compounds as Corrosion Inhibitors

The inventive compounds were tested as corrosion inhibitors in the Shell wheel test. Coupons of carbon steel (DIN 1.1203 with surface area 15 cm²) were immersed into a saltwater/petroleum mixture (9:1.5% NaCl solution adjusted to pH 3.5 with acetic acid) and exposed to this medium at a peripheral speed of 40 rpm at 70° C. for 24 hours. The dosage of the inhibitor was 50 ppm of a 40% solution of the inhibitor. The protection values were calculated from the mass decrease of the coupons based on a blank value.

In the tables which follow, “comparative 1” denotes a commercial residue amine quat based on dicocoalkyldimethylammonium chloride and “comparative 2” an example from JP 49026145 (N-lauroylglycine sodium salt, prior art corrosion inhibitor), and “comparative 3” an example from JP-8 337 562 (N-myristoyl-L-aspartic acid disodium salt, prior art corrosion inhibitor).

TABLE 1
(Shell wheel test)

| | | Ø |
|---|---|---|
| Example | Corrosion inhibitor | % protection |
| comparative 1 | standard quat | 36 |
| comparative 2 | N-lauroylglycine sodium salt | 45 |
| comparative 3 | N-myristoyl-L-aspartic acid disodium salt | 38 |
| 9 | compound from example 1 | 63 |
| 10 | compound from example 2 | 72 |
| 11 | compound from example 3 | 78 |
| 12 | compound from example 4 | 80 |
| 13 | compound from example 5 | 85 |
| 14 | compound from example 6 | 83 |
| 15 | compound from example 7 | 82 |
| 16 | compound from example 8 | 85 |

The products were also tested in the LPR test (test conditions analogous to ASTM D 2776).

TABLE 2
(LPR test)

| | | Protection after [%] | | |
|---|---|---|---|---|
| Example | Corrosion inhibitor | 10 min | 30 min | 60 min |
| comparative 1 | standard quat | 53.9 | 61.2 | 73.7 |
| comparative 2 | N-lauroylglycine sodium | 15.4 | 35.2 | 42.9 |
| | salt | | | |
| comparative 3 | N-myristoyl-L-aspartic acid | 20.0 | 42.6 | 47.1 |
| | disodium salt | | | |
| 17 | compound from example 1 | 60.5 | 75.3 | 88.4 |
| 18 | compound from example 2 | 62.9 | 76.1 | 90.0 |
| 19 | compound from example 3 | 76.4 | 88.4 | 97.2 |
| 20 | compound from example 4 | 74.8 | 87.3 | 96.8 |
| 21 | compound from example 5 | 90.3 | 94.2 | 98.9 |
| 22 | compound from example 6 | 92.0 | 96.7 | 99.0 |
| 23 | compound from example 7 | 78.5 | 92.9 | 98.5 |
| 24 | compound from example 8 | 80.1 | 94.5 | 98.6 |

As is evident from the above test results, the inventive products have very good corrosion protection properties at low dosage and significantly exceed the effectiveness of the prior art inhibitors.

TABLE 3
(Shaking foam test):

| | | Foaming |
|---|---|---|
| Example | Corrosion inhibitor | behavior |
| comparative 1 | standard quat | highly foaming |
| comparative 2 | N-lauroylglycine sodium salt | highly foaming |
| comparative 3 | N-myristoyl-L-aspartic acid disodium | highly foaming |
| | salt | |
| 17 | compound from example 1 | weakly foaming |
| 18 | compound from example 2 | weakly foaming |
| 19 | compound from example 3 | weakly foaming |
| 20 | compound from example 4 | weakly foaming |
| 21 | compound from example 5 | foaming |
| 22 | compound from example 6 | foaming |
| 23 | compound from example 7 | weakly foaming |
| 24 | compound from example 8 | weakly foaming |

The foam properties were tested by the shaking foam method. To this end, 50 ml of a 3% aqueous solution of the appropriate corrosion inhibitor in demineralized water were shaken 20 times in a closed 100 ml measuring cylinder within 10 seconds. For the assessment of the foaming behavior, after the shaking had ended, the total volume of the solution (foam height) and the foam decay time (time until attainment of the starting volume of 50 ml) were employed. In general, this testing method is moderately reproducible, but is outstandingly suitable for a broad estimation of the foaming behavior into weakly foaming, foaming or highly foaming.

Table 3 shows that the inventive compounds have a significantly lower foam formation tendency than the prior art compounds.

TABLE 4
Biodegradability (OECD 306) and toxicity (EC₅₀ Skeletonema
costatum)

| | | | Toxicity |
|---|---|---|---|
| | | Biodegradability | EC₅₀ |
| Example | Corrosion inhibitor | [%] | [mg/l] |
| comparative 1 | standard quat | 15.2 | 0.57 |
| comparative 2 | N-lauroylglycine sodium | 44.5 | 8.5 |
| | salt | | |
| comparative 3 | N-myristoyl-L-aspartic acid | 50.3 | 9.5 |
| | disodium salt | | |
| 25 | compound from example 1 | 92.4 | 44.5 |
| 26 | compound from example 3 | 84.0 | 22.3 |
| 27 | compound from example 6 | 81.5 | 15.4 |
| 28 | compound from example 8 | 85.4 | 13.6 |

As is clearly evident from table 4, the inventive compounds exhibit a better biodegradability and lower toxicity than the comparative examples from the prior art, especially compared to the standard quat.

1. A corrosion inhibitor comprising the salts of compounds of the formula (1) 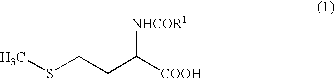
and amines of the formula (2) 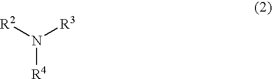
wherein
R¹ is C₁- to C₃₀-alkyl, C₂- to C₃₀-alkenyl, C₆- to C₃₀-aryl or C₇- to C₃₀-alkylaryl,
R² is C₁- to C₃₀-alkyl, C₂- to C₃₀-alkenyl, C₆- to C₃₀-aryl or C₇- to C₃₀-alkylaryl, or an organic radical, optionally containing heteroatoms, and has from 1 to 30 carbon atoms, and
R³, R⁴ are each independently hydrogen, C₁- to C₃₀-alkyl, C₂- to C₃₀-alkenyl, C₆- to C₃₀-aryl or C₇- to C₃₀-alkylaryl, or an organic radical, optionally containing heteroatoms, and has from 1 to 30 carbon atoms, where R³ and R⁴ optionally form a cycle with from 5 to 7 ring atoms including the nitrogen atom.

2. The corrosion inhibitor as claimed in claim 1, wherein R¹ is an alkyl or alkenyl group having from 8 to 18 carbon atoms.

3. The corrosion inhibitor as claimed in claim 1, wherein one, two or all R², R³ and R⁴ radicals are —CH₂—CH₂—OH.

4. The corrosion inhibitor as claimed in claim 1, wherein two of the R², R³, R⁴ radicals are one C₁- to C₈-alkyl group and one —(CH₂CH₂O)ₙ—H group where n=from 2 to 10.

5. The corrosion inhibitor as claimed in claim 1 wherein the amine of the formula (2) is a compound of the formula (3) 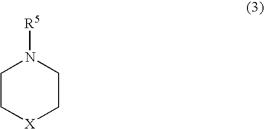
wherein
R⁵ is hydrogen or a C₁₋₃₀ alkyl radical optionally containing heteroatoms, and
X is C, O or N.

6. A device for extraction and transport of hydrocarbons in mineral oil extraction and processing comprising the corrosion inhibitor as claimed in claim 1.

7. A metal processing assistant comprising the corrosion inhibitor as claimed in claim 1.

8. A process for making a salt comprising the step of reacting compounds of the formula (1) 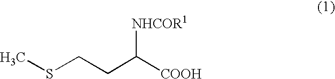
with amines of the formula (2) 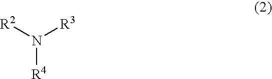
wherein
R¹ is C₂- to C₃₀-alkyl, C₂- to C₃₀-alkenyl, C₆- to C₃₀-aryl or C₇- to C₃₀-alkylaryl,
R² is C₁- to C₃₀-alkyl, C₂- to C₃₀-alkenyl, C₆- to C₃₀-aryl or C₇- to C₃₀-alkylaryl, or an organic radical, optionally containing nitrogen atoms, and has from 1 to 30 carbon atoms, and
R³, R⁴ are each independently hydrogen, C₁- to C₃₀-alkyl, C₂- to C₃₀-alkenyl, C₆- to C₃₀-aryl or C₇- to C₃₀-alkylaryl, or an organic radical, optionally containing heteroatoms, and has from 1 to 30 carbon atoms, wherein R³ and R⁴ optionally form a cycle with from 5 to 7 ring atoms including the nitrogen atom.
